(19) United States
(12) Patent Application Publication
(10) Pub. No.: US 2015/0252329 A1
(43) Pub. Date: Sep. 10, 2015

(54) METHODS AND APPARATUS FOR CULTURING CILIATED CELLS

(71) Applicant: THE UNIVERSITY OF NORTH CAROLINA AT CHAPEL HILL, Chapel Hill, NC (US)

(72) Inventor: Lawrence E. Ostrowski, Durham, NC (US)

(21) Appl. No.: 14/432,987

(22) PCT Filed: Oct. 8, 2013
(86) PCT No.: PCT/US2013/063896

§ 371 Date: Apr. 1, 2015

Related U.S. Application Data
(60) Provisional application No. 61/711,541, filed on Oct. 9, 2012.

(51) Int. Cl. C12N 5/071; G01N 33/50; C12M 1/12

(52) U.S. Cl. CPC C12N 5/0688; C12M 25/02; G01N 33/5044; C12N 2513/00; C12N 2533/30

(57) ABSTRACT

A cell culture assembly includes a cell culturing device and a layer of ciliated cells. The cell culturing device includes at least one member defining an endless, substantially elliptical channel. The layer of ciliated cells is disposed in the elliptical channel.

### RELATED APPLICATION(S)

This application claims the benefit of and priority from U.S. Provisional Patent Application No. 61/711,541, filed Oct. 9, 2012, the disclosure of which is incorporated herein by reference.

### FIELD OF THE INVENTION

The present invention relates to cell culturing devices and methods and, more particularly, cell culturing devices and methods for culturing ciliated cells.

### BACKGROUND

Mucociliary clearance (MCC) is an important innate defense mechanism, and reduced MCC is a component of many airway diseases, including chronic obstructive pulmonary disease (COPD), cystic fibrosis (CF), primary ciliary dyskinesia and asthma. Although there are many studies of the individual components of MCC, there are fewer investigations of the integrated system, in part because of the difficulty of performing MCC studies in vivo. For example, while the effects of many agents on ciliary beat frequency (CBF) have been documented in the literature, the direct effects of agents that stimulate CBF on the rate of MCC are limited. While there are methods in place to measure MCC in whole animals, these techniques are difficult, expensive, and time-consuming. There is also at least one method to study mucociliary transport (MCT) in vitro; however, this method is inconsistent, variable, and also requires significant interpretation.

### SUMMARY OF THE INVENTION

According to embodiments of the present invention, a cell culture assembly includes a cell culturing device and a layer of ciliated cells. The cell culturing device includes at least one member defining an endless, substantially elliptical channel. The layer of ciliated cells is disposed in the elliptical channel.

In some embodiments, the cell culturing device includes a semipermeable membrane forming a floor of the channel and supporting the layer of ciliated cells. In some embodiments, the membrane is porous. The cell culture assembly may include a culture medium in fluid contact with the layer of ciliated cells through the membrane. In some embodiments, the membrane is formed of a material selected from the group consisting of polycarbonate, mixed cellulose esters, polyester and polytetrafluoroethylene (PTFE). Reference indicia may be provided on the membrane to assist in measuring a rate of ciliary transport by the layer of ciliated cells.

According to some embodiments, the cell culture assembly includes a side wall and reference indicia on the side wall to assist in measuring a rate of ciliary transport by the layer of ciliated cells.

In some embodiments, the cell culture assembly includes at least one tracking bead on the layer of ciliated cells to assist in measuring a rate of ciliary transport by the layer of ciliated cells.

The cell culture assembly may be configured such that the ciliated cells spontaneously orient themselves to transport a material on the layer of ciliated cells in a continuous pattern around the elliptical channel.

According to some embodiments, the layer of ciliated cells is a layer of ciliated airway epithelial cells. In some embodiments, the cell culture assembly includes a layer of a transport fluid on top of the layer of ciliated airway epithelial cells. In some embodiments, the cells are human bronchial epithelial cells. In some embodiments, the transport fluid is mucus.

According to some embodiments, the channel has a substantially uniform width about its full circumference.

In some embodiments, the cell culturing device includes a floor of the channel supporting the layer of ciliated cells, and the floor has a curved cross-sectional profile.

According to method embodiments of the present invention, a method for culturing ciliated cells includes: providing a cell culturing device including at least one member defining an endless, substantially elliptical channel; and placing a layer of ciliated cells in the elliptical channel.

In some embodiments, the cell culturing device includes a semipermeable membrane forming a floor of the channel, and the method includes placing the layer of ciliated cells on the membrane such that the layer of ciliated cells is supported thereby. The method may include providing a culture medium in the cell culturing device and contacting the layer of ciliated cells with the culture medium through the membrane.

According to some embodiments, the ciliated cells spontaneously orient themselves to transport a material on the layer of ciliated cells in a continuous pattern around the elliptical channel.

The method may include using the cell culturing device to screen, test, examine, and/or compare the effectiveness of one or more drugs and/or chemical agents for their effect on improving mucociliary transport by the layer of ciliated cells.

According to embodiments of the present invention, a cell culturing device for culturing a layer of cells includes at least one member defining an endless, substantially elliptical channel, and a semipermeable membrane forming a floor of the channel to receive and support the layer of cells.

In some embodiments, the cell culturing device is configured such that, when the layer of cells is a layer of ciliated cells, the cell culturing device causes the ciliated cells to spontaneously orient themselves to transport a material on the layer of cells in a continuous pattern around the elliptical channel.

According to method embodiments of the present invention, a method for culturing ciliated cells comprising: culturing ciliated cells in a cell culturing device; and using the cell culturing device to screen, test, examine, and/or compare the effectiveness of one or more drugs and/or chemical agents for their effect on improving mucociliary transport by the layer of ciliated cells.

Further features, advantages and details of the present invention will be appreciated by those of ordinary skill in the art from a reading of the figures and the detailed description of the embodiments that follow, such description being merely illustrative of the present invention.

### BRIEF DESCRIPTION OF THE DRAWINGS

FIG. 1 is a top, plan view of an MCT culturing device (MCTD) according to embodiments of the present invention.

FIG. 2 is a cross-sectional view of the MCTD of FIG. 1 taken along the line 2-2 in FIG. 1.

FIG. 3 is a fragmentary, top plan view of a culturing system, including the MCTD of FIG. 1, and a layer of ciliated cells plated in the MCTD to form a cell culture assembly.

FIG. 4 is a cross-sectional view of the cell culture assembly of FIG. 3 taken along the line 4-4 of FIG. 3 and a heater block.

FIG. 5 is a flow chart representing methods according to embodiments of the present invention.

FIG. 6 is a fragmentary, top plan view showing the movement of fluorescent beads in the airway surface liquid of HAE cells cultured in the MCTD.

FIG. 7 is a graph representing experimental overall average rate of MCT for four HAE cell cultures each plated in a respective MCTD.

FIG. 8 is a graph representing the effect of decreasing temperatures on CBF and MCT speed as experimentally measured using the MCTD.

FIG. 9 is a graph representing the effect of increasing temperatures on CBF and MCT speed as experimentally measured using the MCTD.

FIG. 10A is a graph representing the effect of temperature on CBF and MCT speed as experimentally determined by raising the temperature of the cell culture step-wise in the MCTD.

FIG. 10B is a graph wherein the data of FIG. 10A is replotted to demonstrate the linear relationship between CBF and MCT speed.

FIGS. 11, 12A and 12B are graphs showing the effect on CBF and MCT speed of stimulation by ATPγS of HAE cells cultured in the MCTD.

FIG. 13 is a graph representing the effect of Bovine Salivary Mucin (BSM) concentration on the MCT speed of HAE cells cultured in the MCTD.

FIG. 14 is a cross-sectional view of an MCTD according to further embodiments of the present invention.

### DETAILED DESCRIPTION OF EMBODIMENTS OF THE INVENTION

The present invention now will be described more fully hereinafter with reference to the accompanying drawings, in which illustrative embodiments of the invention are shown. In the drawings, the relative sizes of regions or features may be exaggerated for clarity. This invention may, however, be embodied in many different forms and should not be construed as limited to the embodiments set forth herein; rather, these embodiments are provided so that this disclosure will be thorough and complete, and will fully convey the scope of the invention to those skilled in the art.

It will be understood that, although the terms first, second, etc. may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the teachings of the present invention.

Spatially relative terms, such as “beneath”, “below”, “lower”, “above”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the exemplary term “below” can encompass both an orientation of above and below. The device may be otherwise oriented (rotated 90° or at other orientations) and the spatially relative descriptors used herein interpreted accordingly.

As used herein, the singular forms “a”, “an” and “the” are intended to include the plural forms as well, unless expressly stated otherwise. It will be further understood that the terms “includes,” “comprises,” “including” and/or “comprising,” when used in this specification, specify the presence of stated features, integers, steps, operations, elements, and/or components, but do not preclude the presence or addition of one or more other features, integers, steps, operations, elements, components, and/or groups thereof. It will be understood that when an element is referred to as being “connected” or “coupled” to another element, it can be directly connected or coupled to the other element or intervening elements may be present. As used herein, the term “and/or” includes any and all combinations of one or more of the associated listed items.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of this specification and the relevant art and will not be interpreted in an idealized or overly formal sense unless expressly so defined herein.

Numbers presented in parentheses represent citations to the references listed at the end of the Detailed Description of the Embodiments of the Invention.

Methods and apparatus according to embodiments of the present invention can provide a simple, reproducible device for studying mucociliary transport (MCT) in vitro. According to embodiments of the invention, ciliated cells are cultured in a cell culturing device (the MCT culturing device) having a closed elliptical (according to some embodiments, circular) channel defining a corresponding elliptical track. Preliminary studies have demonstrated that culturing ciliated airway epithelial cells in the channel of a MCT culturing device as described will result in the development of MCT in a continuous fashion along the track and around the MCT culturing device. A device as disclosed can have broad applications to both basic scientific studies of the process of MCT and to the development and testing of therapeutics designed to improve mucociliary clearance (MCC) in a wide range of airway diseases, including chronic obstructive pulmonary disease (COPD), cystic fibrosis (CF), and asthma (Fahy and Dickey 2010).

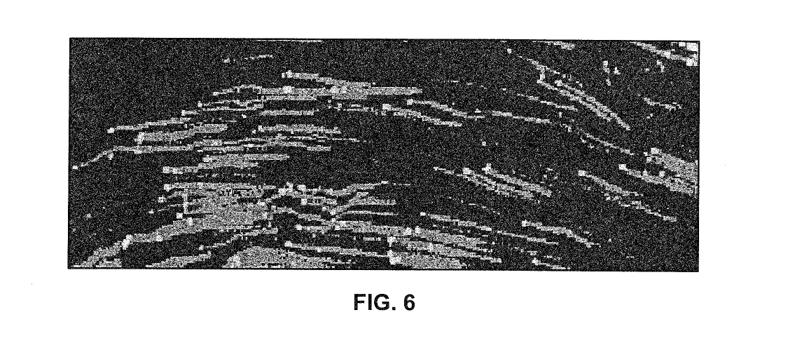

With reference to FIG. 5, a method or process for culturing ciliated cells in accordance with embodiments of the invention includes providing a cell culturing device defining an endless, substantially elliptical channel (Block 20), and placing or culturing a layer of ciliated cells in the elliptical channel (Block 22). The culturing device may be constructed as described below. The placed ciliated cells may spontaneously orient themselves to transport material (e.g., mucus) on the layer of ciliated cells in a continuous pattern around the elliptical channel. The cell culturing device may be further used to screen, test, examine and/or compare the effectiveness of one or more drugs or chemical agents for their effect on MCT.

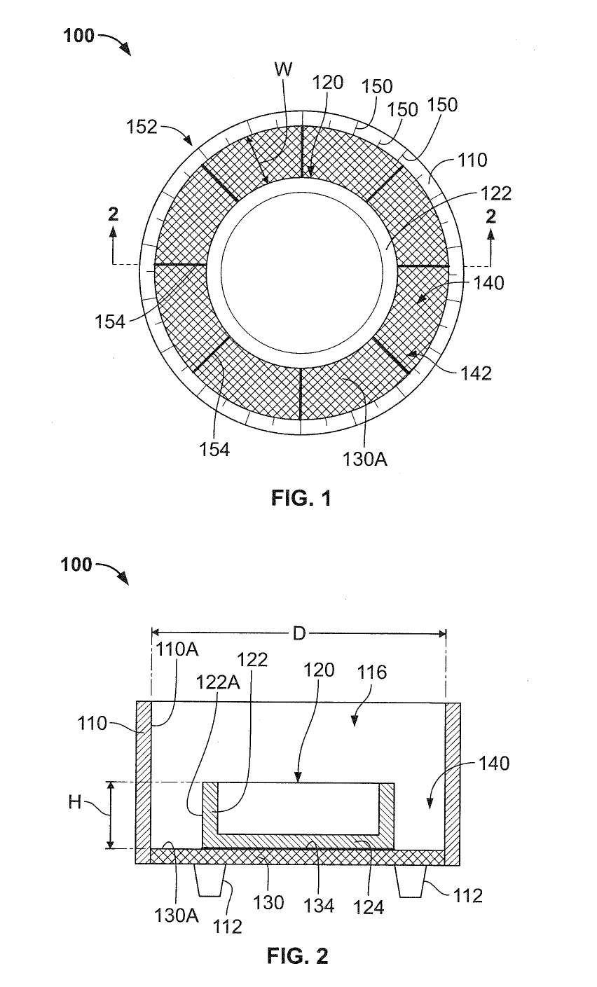

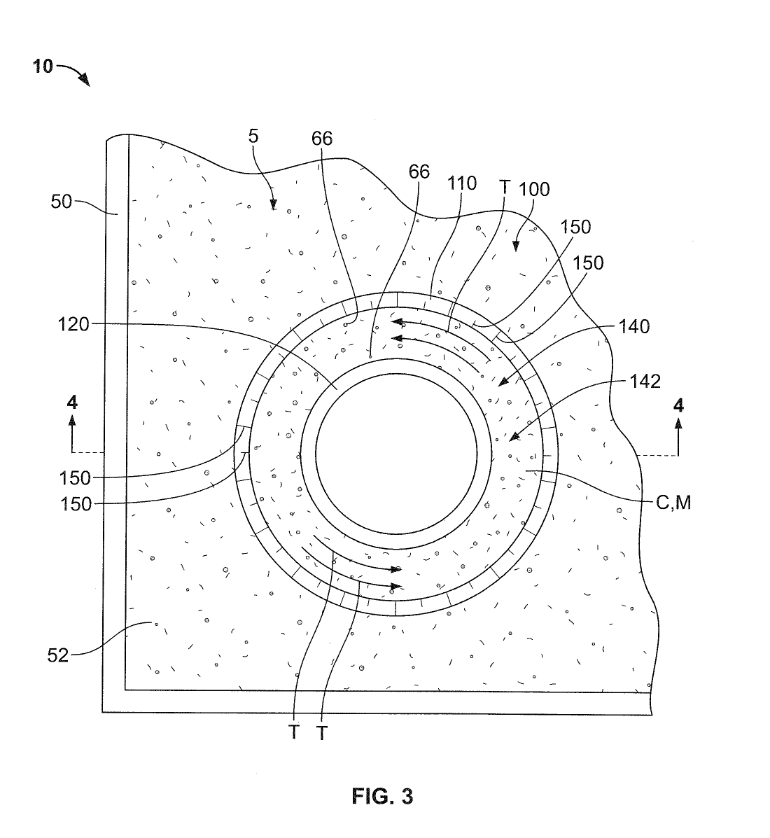

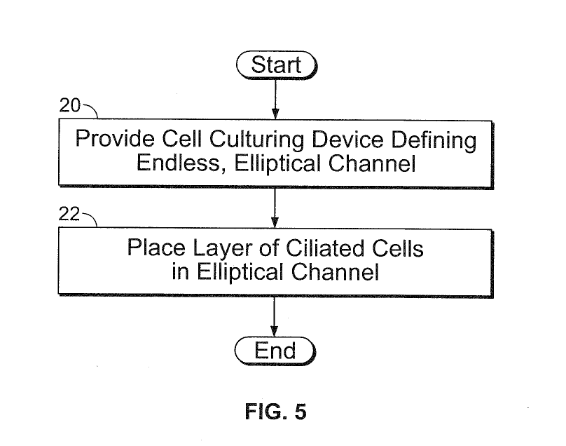

With reference to FIGS. 1-4, an exemplary MCT culturing device (MCTD) 100 according to embodiments of the present invention is shown therein. The MCT culturing device 100 may be used in combination with a culturing vessel 50 and a culture medium 52, which collectively form a culturing system (MCTS) 10. The MCTS system 10 may further include a heater block 60 and a heater block cover 62 (FIG. 4) to selectively and controllably heat the MCT culturing device 100. The MCTS system 10 may also include a video camera and microscope 64 for capturing and recording movement in the MCT culturing device 100.

The MCT culturing device 100 includes an annular or cylindrical outer wall or member 110, an inner member 120, and a floor member 130. The outer member 110 and the floor member 130 define a cavity 116 in which the inner member 120 is disposed. The inner member 120 includes a bottom wall 124 and an upstanding, annular or cylindrical sidewall 122. The bottom wall 124 can be bonded to the floor member 130 by a layer of adhesive 134. A plurality of support or spacer feet 112 depend from the outer member 110.

An annular inner wall surface 110A of the outer member 110, an opposing annular wall surface 122A of the sidewall 122, and an annular surface section 130A of the floor member 130 collectively define an annular channel 140. According to some embodiments and as shown, the channel 140 is continuous, is closed or endless, and is elliptical in shape. According to some embodiments, the channel 140 is substantially circular in shape (a circle being a special kind of ellipse). According to some embodiments, the surfaces 110A and 122A are substantially concentric. The channel 140 defines a track 142 for MCT, as discussed herein.

According to some embodiments, the channel 140 has a width W in the range of from about 0.5 mm to 6 mm and, according to some embodiments, the channel width W is in the range of from about 0.5 mm to 2 mm. According to some embodiments, the channel width W is substantially uniform about the entire length or circumference of the channel 140. According to some embodiments, the channel 140 has a height H of at least about 5 mm. According to some embodiments, the channel 140 has an outer diameter D in the range of from about 2 mm to 30 mm.

According to some embodiments and as shown, the floor member 130 is a semipermeable or porous membrane. The membrane may be formed of any suitable material(s). According to some embodiments, the membrane is formed of a material selected from the group consisting of polycarbonate, mixed cellulose esters, polyester and polytetrafluoroethylene (PTFE). The membrane may be coated with or incorporate collagen or other matrices for the culturing of the cells.

The inner and outer members 110, 120 may be formed of any suitable material(s). According to some embodiments, the members 110, 120 and the surfaces 110A and 122A are formed of a rigid polymeric material. According to some embodiments, the polymeric material is or includes polystyrene or nylon.

The culturing vessel 50 and the culture medium 52 may be of known, conventional or any other suitable type and construction. Such devices and mediums are well known to those of skill in the art and will not be described in detail herein. According to some embodiments, the MCT culturing device 100 and the culturing vessel 50 are relatively sized so that multiple MCT culturing devices 100 can be placed in side-by-side arrangement in the vessel 50 to enable high throughput screening (HTS), for example. The vessel 50 may be a flask, a culture dish, or a 6, 24, 96 or 384 well culture plate, for example.

In some embodiments, the MCT culturing device 100 includes a 30 mm diameter outer member 110 surrounding an approximately 18 mm diameter inner wall 122. This results in a circular membrane track 142 with a width W of about 4 to 5 mm. This size MCT culturing device 100 can be used in a standard 6-well culture plate 50. However, the size of the device 100 can be modified to fit any standard cell culture vessel, including, but not limited to, 12, 24, 48, 96, and 384 well-plates.

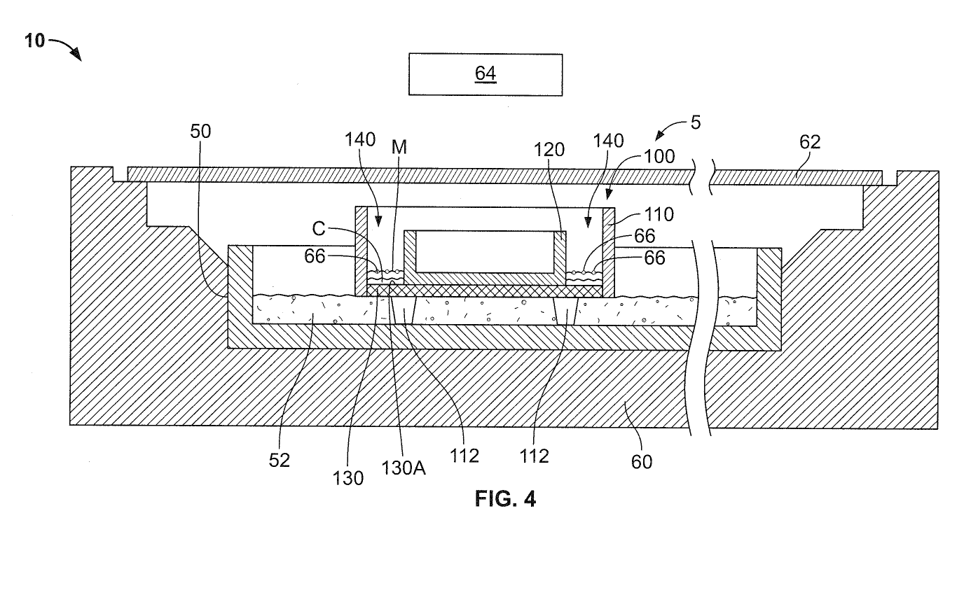

An MCT culturing device (e.g., the MCT culturing device 100) and a culturing system (e.g., the culturing system 10) may be used as follows in accordance with embodiments of the present invention to induce and observe MCT in a ciliated cell sample. With reference to FIGS. 3 and 4, a sample or layer of ciliated cells C are seeded, plated or placed on the membrane surface 130A of the MCT culturing device 100 to form a cell culture assembly 5. According to some embodiments, which may include each of the embodiments discussed and disclosed herein, the cells C are ciliated cells. A layer of transport fluid M is disposed on top of the layer of cells C. In some embodiments, the transport fluid M is endogenous mucus. In other embodiments, the transport fluid M is exogenous (added) mucus.

The device 100 is placed in the vessel 50 such that the culture medium 52 contacts at least the bottom or basolateral side of the membrane 130 (e.g., the upper level of the culture medium 52 is at or above the height of the membrane 130). The layer of transport fluid M may be exposed to the overlying air or other atmosphere as shown. According to some embodiments, the device 100 and method position and contain the ciliated cells C in an orientation that causes the ciliated cells C to organize their activity in a synchronous fashion, so that the layer of ciliated cells C thereby transports the fluid M in a pre-defined, circular pattern or direction T in the channel 140 and along the track 142. The porous membrane 130 material supports the growth and differentiation of the ciliated cells C. The ciliated cells C, being cultured at the air/liquid interface, are nourished by the culture medium 52 which is in fluid contact with the ciliated cells C.

According to some embodiments, the ciliated cells C are epithelial cells and, in some embodiments, are human airway epithelial (HAE) cells. It has been found that ciliated epithelial cells, when cultured in the described MCT culturing device 100, will spontaneously orient themselves in such a manner as to transport materials in a continuous circular pattern that can be measured and studied.

In some embodiments, an MCT culturing device (e.g., the device 100) as described herein is used for screening agents to improve MCC. Therapeutic agents or treatments known to increase/decrease ciliary beat frequency (CBF) are tested using the MCT culturing device for their ability to increase/decrease the rate of transport in the MCT culturing device.

Impaired MCC is an important contributing factor to many debilitating airway diseases, including CF and COPD (a chronic disease that affects more than 200 million people). Efficient mucociliary clearance (MCC) is the result of the coordinated regulation of multiple cellular systems, including the production and secretion of mucin, the regulation of ion and fluid transport, and the coordinated beating of cilia. In cystic fibrosis (CF), inactivating mutations in the CFTR protein prevent the secretion of chloride onto the apical surface of the airways. As a result, the mucous layer becomes dehydrated and MCC is impaired. The reduction in MCC renders the CF individual more susceptible to viral and/or bacterial infections. Infections cause an increase in mucin secretion, and this leads to a further dehydration of the airway surface liquid. While normal mucus may contain approximately 2% solids, CF mucus typically contains 6-8% solids, which studies have shown results in the collapse of the periciliary layer and a complete inhibition of MCC (Button, Cai et al. 2012). Thus, there is strong evidence that reduced MCC plays a key role in the initiation and progression of CF lung pathogenesis. The role of MCC in CF lung disease is further evidenced by clinical studies that have demonstrated that agents that improve MCC also improve patient outcome. Thus, inhaling DNase, which is proposed to reduce mucus viscosity and improve MCC by cleaving the abundant DNA found in CF mucus, has been a standard of care for almost 20 years (Wagener and Kupfer 2012). More recently, the inhalation of hypertonic saline (Donaldson, Bennett et al. 2006) or mannitol (Aitken, Bellon et al. 2012) to rehydrate the airway surface and improve MCC has been demonstrated to improve lung function in CF patients. However, neither of these agents is completely effective at restoring MCC and preventing disease progression in CF, and it is clear that improved treatments are still necessary.

While there have been many studies of the individual components of MCC, including the regulation of mucus secretion (Davis and Dickey 2008), ciliary beat frequency (Salathe 2007), and airway liquid secretion/absorption (Boucher 2003), there have been far fewer studies of MCC. This is in large part due to the difficulties of studying an integrated system that involves multiple cell types and directional transport. Thus, most studies of MCC to date have been performed using whole animal models that require specialized techniques and have limited throughput (Foster, Walters et al. 2001; Sabater, Wanner et al. 2002; Grubb, Jones et al. 2004; Hua, Zeman et al. 2010). For this reason, our understanding of the detailed mechanisms controlling efficient MCC are limited, and the development of new therapeutics to improve MCC has also been hampered.

The development of a simple, reproducible device (namely, an MCT culturing device as described herein in accordance with embodiments of the present invention) will allow for detailed in vitro studies of the MCT system and may stimulate research in this area. In addition, the MCT culturing device and system as disclosed herein can allow for relatively high-throughput screening of potential therapeutic agents, either singly or in combination, including agents that increase CBF (e.g., salmeterol (Bennett, Almond et al. 2006)), decrease mucus production (e.g., azithromycin (Ribeiro, Hurd et al. 2009)) or viscosity (e.g., N-acetylcysteine (Suk, Boylan et al. 2011)), or increase mucus hydration (e.g., amiloride (Zhou, Treis et al. 2008)).

One of the difficulties with developing agents to improve MCC is that, as mentioned above, MCC is the result of the complex interplay of several different components. And while there are many assays that are capable of measuring the effect of potential therapeutic agents on individual components of MCC (e.g., mucus viscosity (Rancourt, Tai et al. 2004), ciliary beat frequency (Morse, Smullen et al. 2001), etc.) there are fewer assays that directly measure the effect on the overall system. Typically, measurement of MCC requires the use of radioactive tracers in humans or large animals (e.g., sheep) (Donaldson, Corcoran et al. 2007; Hirsh, Zhang et al. 2008). These assays are expensive and the number of compounds that can be tested are limited. Although a few laboratories have measured MCC in mouse models (Grubb, Jones et al. 2004; Hua, Zeman et al. 2010; Ostrowski, Yin et al. 2010), these studies are labor intensive and still require the use of animals.

In in vitro studies of MCT, it has been observed that well-differentiated cultures of human bronchial epithelial (HBE) cells grown at the air/liquid interface (ALI) occasionally develop areas of circular mucociliary transport (MCT) (Matsui, Grubb et al. 1998; Matsui, Randell et al. 1998). These “mucus hurricanes” have been very useful to researchers in the field, and have been important to our developing understanding of MCC (Tarran and Boucher 2002; Tarran 2004). However, these cultures suffer from several disadvantages that make them less than ideal for purposes of drug screening/development. For example, the development of these areas of MCT is occasional, rather than reproducible, and the size and location of the MCT is random within the culture dish. More importantly, because of the orientation of these areas of MCT, mucus tends to accumulate in the center, and the speed of transport depends on the distance from the center of “hurricane”. Thus this system is inadequate for the standardized testing and comparison of the effect of novel agents on MCT. The lack of a suitable in vitro system for MCT studies represents a critical gap in our ability to develop new therapeutics targeting this innate defense mechanism.

MCT culturing devices and systems as disclosed herein (e.g., the MCT culturing device 100 and the culturing system 10) can provide continuous, directional (elliptical or substantially circular) MCT in a uniform and reproducible manner. It is believed that this phenomena results from the inventive MCT culturing device because the cultured cilia will spontaneously beat in the direction of least resistance and organize into a metachronal wave capable of transporting mucus. Applying this concept, airway epithelial cells are cultured in a relatively narrow, ring-shaped channel so that the path of least resistance for MCT is around the culture channel in a circular pattern that encompasses the entire circumference of the MCT culturing device.

The MCT culturing device results in a more reproducible generation of MCT in vitro, because the ciliated cells are “directed” or “guided” to organize their ciliary activity in a pre-defined circular pattern. A plurality of such MCT culturing devices may be provided having a uniform width and diameter, which can allow for the standardization of measurements of MCT and thus increase the ease and speed of measurements. The MCT culturing device can also allow multiple repeat measures of the same cells under the same conditions, because once MCT is organized, the physical constraints of the MCT culturing device will make it unlikely that the pattern of MCT will change. The MCT culturing device can also be adapted for HTS of new therapeutic agents, which is not currently feasible using other techniques to study MCT.

According to some method embodiments of the present invention, an MCT culturing device as disclosed herein is employed as follows. Human airway epithelial cells are plated in the track and cultured at the air-to-liquid interface (ALI) using standard conditions until ciliated cell differentiation occurs (Gray, Guzman et al. 1996; Fulcher, Gabriel et al. 2005). The cultures are monitored for the spontaneous development of MCT in a circular pattern around the track. The number of cultures that develop MCT and the direction of flow (clockwise (CW) or counterclockwise (CCW)) is determined. Marker or tracking beads can be added to the mucus layer and tracked or monitored and recorded to measure the rate of MCT. In particular, fluorescent beads may be added to the mucus layer and the rate of MCT can be measured by fluorescent microscopy (Worthington and Tarran 2011). The rate of MCT may also be measured by visually observing masses (“globs”) of endogenous mucus and/or debris move around the track without added fluorescent beads.

The size and dimensions of the MCT culturing device may be varied to improve its usefulness. For example, a narrower track (e.g., the track 142) may increase the number of cultures that successfully generate circular MCT. The MCT culturing device may also be scaled down to 24, 96 and/or 384 well formats so that it can be utilized for HTS of therapeutic agents. Reducing the size of the MCT culturing device can provide other advantages, including a decreased cost of production and the use of fewer cells and reagents.

An external flow may be applied to the cell culture in the MCT culturing device during the differentiation period to orient the direction of MCT. The application of flow may increase the success rate of generating circular MCT.

MCT culturing devices as described herein may also be used to culture airway epithelial cells from mice (You, Richer et al. 2002), dogs (Kondo, Finkbeiner et al. 1991), pigs, or other vertebrate animal species. Such expanded utility of the MCT culturing device may be valuable because mouse, dog, and porcine cells are more readily available than human cells. In addition, the ability to study MCT using cells from genetically modified mouse lines would be a further advantage of the system.

As noted above, it has been previously observed that well-differentiated cultures of human bronchial epithelial (HBE) cells grown at the air/liquid interface (ALI) occasionally develop areas of circular mucociliary transport (MCT) (Matsui, Grubb et al. 1998; Matsui, Randell et al. 1998). However, the random occurrence of these hurricanes limits their usefulness as a screening assay. Because there is evidence that cilia can spontaneously coordinate their beat pattern (as evidenced by the formation of mucus hurricanes), and that cilia “prefer” to beat in the direction of least resistance, the inventors hypothesized that if placed in a narrow, circular track, the developing cilia would self-organize to produce continuous, directed, mucociliary transport. This circular track is believed to have several advantages: 1) because the track is narrow, cilia communication occurs over shorter distances than in a large culture dish; 2) because the track is defined by two edges, the cilia are “directed” to beat in either a plus or minus direction around the track; 3) the mucus is be transported around the track, therefore it does not accumulate; and 4) the flow of mucus around the track further refines the coordination of the cilia.

### Example 1

An MCT culturing device (MCTD) and the MCT system (MCTS) generally configured as shown for the MCTD 100 and MCTS 10 were constructed. The MCTD was constructed on a 30 mm Millicell™ cell culture insert (Millipore, Ireland). The base of an approximately 15 mm diameter central cylinder of cell culture compatible material (nylon, polystyrene) was attached to the center interior of the cell culture insert with an inert, silicone-based sealer. The approximately 4.0 mm outer ring or track defined in the bottom of the cell culture insert between the central cylinder and the cell culture insert was then coated with human Type IV collagen at ˜90 μg/cm², air-dried, and UV-irradiated. All subsequent cell culture procedures were carried out essentially as previously described in detail (Gray, Guzman et al. 1996; Fulcher, Gabriel et al. 2005), with the following modifications. Primary or passage 1 HBE cells prepared by the University of North Carolina Cell and Tissue Culture Facility were plated on the MCTD (i.e., in the track; 500,000 or 250,000 cells/cm², respectively) and fed basally with ALI media, 3×/week, for the first two weeks of culture. For the remainder of the experiment, cultures were routinely fed twice weekly. Cultures were washed to remove mucus and cell debris at each media change with 1-2 ml of phosphate buffered saline (PBS) that was added to the apical surface and allowed to incubate for 5 minutes. Cultures were examined at the time of media change and the extent of ciliation and the development of continuous, circular transport was recorded. Typically, cultures developed continuous MCT after approximately six weeks of culture. Not all cultures developed continuous circular MCT. Many cultures developed large areas of transport (½ to ⅔ of the culture dish), but failed to transport completely around the culture. Some cultures “dehydrated” between media changes and only exhibit complete MCT after the PBS wash. The inventors believe that a portion of this variability is dependent on the variability in the donor cells. There also is an apparent lot-to-lot variability in the Millicell™ cell culture inserts used to construct the device.

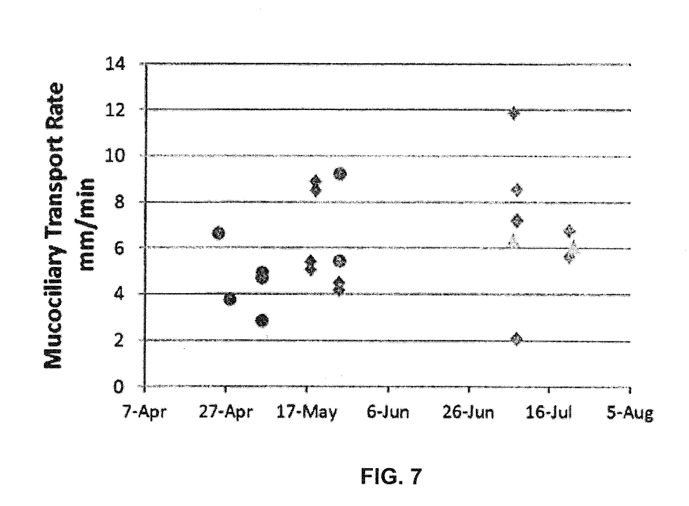

Briefly, to perform a typical MCT experiment, the MCTD was washed and refed on day 0 as described above (although the details can vary, depending on the goal of the experiment). The following day, the culture was examined for the presence of MCT. If present, the insert was placed in a culture dish containing HEPES buffer and transferred to a heating block on the stage of a Nikon™ TE-2000 microscope. High speed videos were recorded from four equally spaced positions around the MCTD. Ciliary beat frequency (CBF) was determined by whole field analysis using the SAVA™ software program (Sisson, Stoner et al. 2003), and the speed and direction of particle transport were determined by tracking particles of debris in the mucus. Alternatively, a small volume of fluid (0.5 μl) containing fluorescent beads was added to the mucus layer, and after equilibration (5-10 minutes), the motion of the fluorescent beads was recorded and analyzed using fluorescent microscopy. Typically, the speed of 3-5 particles was measured in each field at each time point and averaged. As shown in FIG. 6, the addition of fluorescent beads to the endogenous mucus layer provided an easy method for measuring the rate of MCT. FIG. 6 is a prolonged-exposure image showing the movement of 3 μm fluorescent beads (light grey) in the airway surface liquid (ASL) of human airway epithelial cells cultured in the inventive MCT culturing device. The motion of each particle is shown as a medium grey “track”. The image was recorded over approximately 7 seconds.

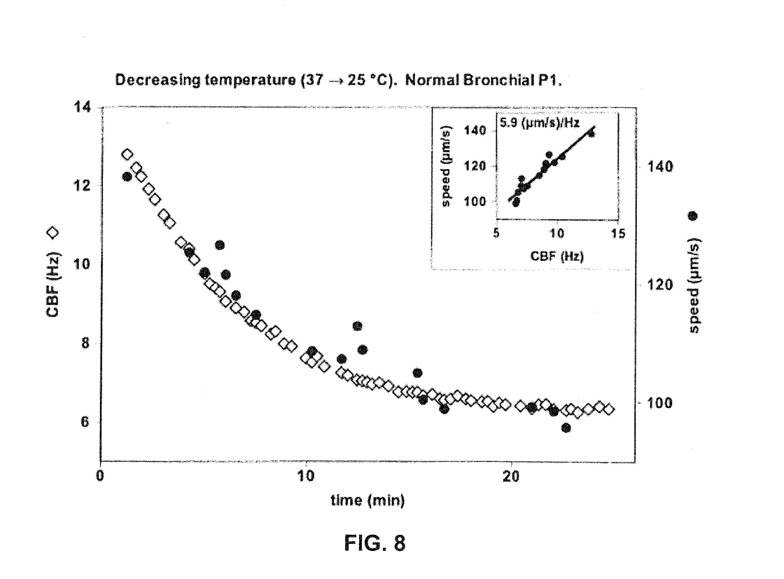

Mucociliary transport around the track was routinely observed in the cultures. Some cultures did not develop MCT around the entire dish, most likely due to imperfections in the formation of the track (i.e., excess sealer disrupting the flow). Mucociliary transport rates were determined by recording the movement of fluorescent beads with a fluorescent microscope and measuring the distance traveled over time. Measurements were obtained using several different size beads (1, 3, or 10 μm) on multiple days. FIG. 7 shows data collected from several MCTD cultures exhibiting circular MCT obtained from several different donors (codes) over time. The data shows that once established, circular MCT can be maintained for periods of several weeks and that the rate of MCT is relatively constant, both between different codes and between measures of the same code.

The overall average rate of MCT from these four cultures was 6.2 mm/min, which agrees well with estimates of in vivo MCC rates (Grubb, Jones et al. 2004; Ostrowski, Yin et al. 2010). In FIG. 7, each symbol represents cultures from a different donor, and each point represents a single culture. Importantly, one of the cultures included in this data (triangles) was obtained from a CF donor, and demonstrates that when cultured under these in vitro conditions, CF cells are also capable of developing continuous MCT. All other cultures were non-CF controls.

According to further method embodiments, studies as described above may be executed as follows. Multiple MCT culturing devices plated with human ciliated cells are cultured and examined under identical or substantially the same conditions. The method may be used to establish the reproducibility of the inventive culturing method or technique and to establish the normal baseline rate of MCT in the MCT culturing system.

In an exemplary method as described, circular cultures of human bronchial epithelial (HBE) cells that have developed MCT are washed (e.g., 3× with phosphate buffered saline (PBS)) to remove mucus and accumulated cell debris. After a prescribed time period (twenty-four hours later), tracking media (e.g., 2 μL of a suspension of 10 micron fluorescent polystyrene beads (Polysciences, Inc.)) is carefully added to the apical fluid layer with a micropipette. The culture is placed on the stage of a fluorescent microscope and the transport of the tracking media (beads) is recorded on an attached DVD recorder. The video is analyzed by measuring the time required for individual tracking media (i.e., bead particles) to travel a fixed distance (determined with a scale bar). The rate of MCT for each MCT culturing device can be determined from the average of at least a prescribed number (e.g., five) different particles measured within a prescribed time period (e.g., the first five minutes after addition of the beads).

According to methods of the present invention, to determine the effect of the width of the channel or track on the development of MCT, MCT culturing devices having channels with different sized widths W (FIG. 1) are constructed and provided. Human airway epithelial cells from different donors are each plated on an MCT culturing device of each size, cultured as above, and the incidence and rate of MCT measured.

To test the effect of externally applied flow on the development of MCT, HBE cells may be cultured on an MCT culturing cell as described herein. At the time ciliated cell differentiation is first evident, a small volume of PBS (e.g., approximately 2 ml) is added to the surface of the culture dish. The dish is then swirled gently for a period of time (e.g., two minutes) in either the CW or CCW direction and the PBS removed. This process is repeated daily for a period of time (e.g., over the next two weeks or until MCT is observed). In each experiment, one or more cultures are swirled CW, one or more cultures are swirled CCW, and multiple cultures are stationary. The time at which MCT develops and the direction of flow will be recorded for each MCT culturing device. Flow may also be generated by using a variety of devices to rotate, rock, or shake the MCT device in a regular pattern to generate a directional flow of fluid. In some embodiments, directional flow may also be applied by a system of tubing and pumps. The application of directional flow may increase the number of MCT devices that establish MCT and/or improve the extent of MCT in individual devices.

Similarly, airway epithelial cells from other mammalian sources such as mice, dogs, or pigs (e.g., cultures of mouse, dog, or pig bronchial or tracheal epithelial cells) may be cultured and studied as above.

According to some method embodiments, treatments known to increase/decrease ciliary beat frequency (CBF) are tested for their ability to increase/decrease the rate of transport in the MCT culturing device to calibrate or validate the usefulness of the MCT culturing device for screening agents to improve MCC. Differentiated cultures of airway epithelial cells that are demonstrating active MCT undergo treatments to increase/decrease CBF and the effect on MCT is measured. These studies can be used to demonstrate that MCT in the closed circular culture track of the MCT culturing device can be regulated similarly to the in vivo situation.

### Example 2

To determine if the rate of MCT around the track could be regulated, ciliary beat frequency (CBF) and the rate of MCT were measured in a single culture immediately after removal from the cell culture incubator (˜37° C.). The cell culture media in the basal compartment of the culture dish was then replaced with cold media (˜0.4° C.) and the measurements were repeated. As shown in Table 1 (showing the effect of low temperature on CBF and MCT in a circular culture of HAE cells), the change to cold temperature reduced the CBF from 13.0 to 4.2 Hz, while the rate of MCT was reduced from 12.4 to 6.7 mm/min. This data demonstrates the rate of MCT can be regulated, and therefore the device can be utilized to identify agents that improve MCT.

TABLE 1

| | Ciliary Beat | Mucociliary |
|---|---|---|
| | Frequency | Transport |
| At 37 degree | 13.0 | 12.4 mm/min |
| After 4 degree | 4.2 | 6.7 mm/min |
| media change | | |

### Example 3

In a further application of the MCTD to study factors affecting MCC, the inventors investigated the relationship between ciliary beat frequency and the rate (speed) of MCT as follows using HAE cells cultured in the MCTD as described. Multiple experiments were performed in which CBF and the rate of MCT was simultaneously measured as the temperature of the culture was varied. Three different protocols were examined: 1) the experiment was started at 37° C. and the culture was allowed to cool to 25° C.; 2) the culture was cooled on ice and the temperature was increased to 37° C.; and 3) the temperature of the culture was increased in a step-wise fashion from 24.5° C. to 39.5° C.

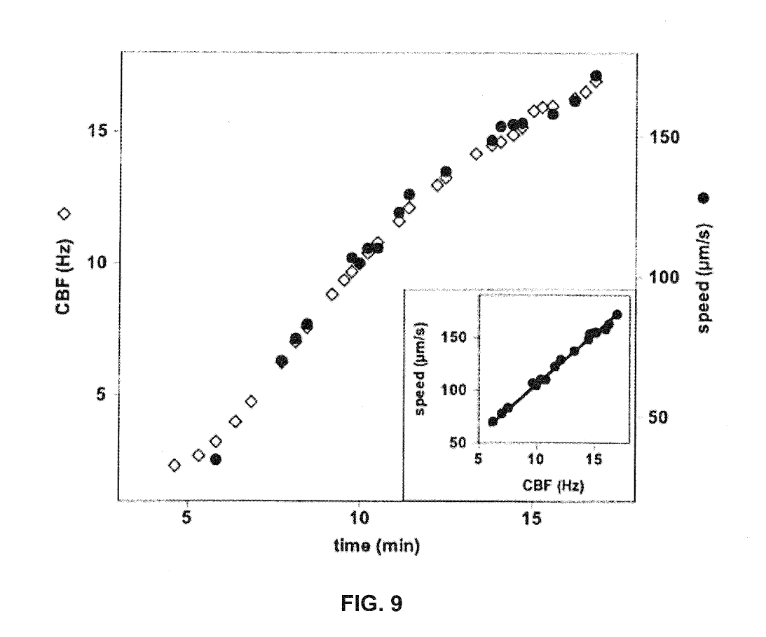

FIG. 8 shows a representative experiment in which HAE cells cultured in the MCTD and demonstrating continuous circular MCT were monitored over time while the culture was allowed to cool from 37° C. to 25° C. CBF and MCT were measured through the observation period and plotted as a function of time. The result shown is from one representative experiment. The insert shows the linear relationship between CBF and MCT.

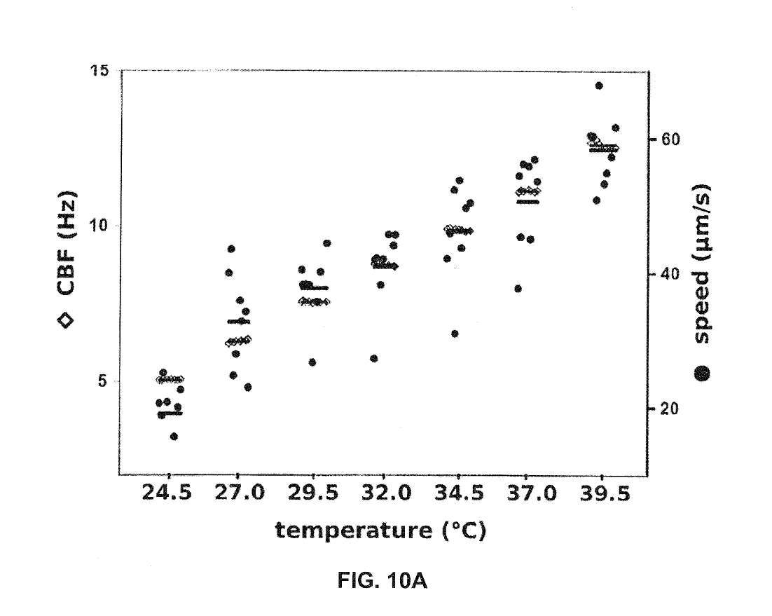

FIG. 9 shows the result of one representative experiment in which HAE cells cultured in the MCTD and demonstrating continuous circular MCT were monitored over time while the temperature was raised from 0° C. to 37° C. CBF and MCT were measured throughout the observation period and plotted as a function of time. The insert shows the linear relationship between CBF and MCT.

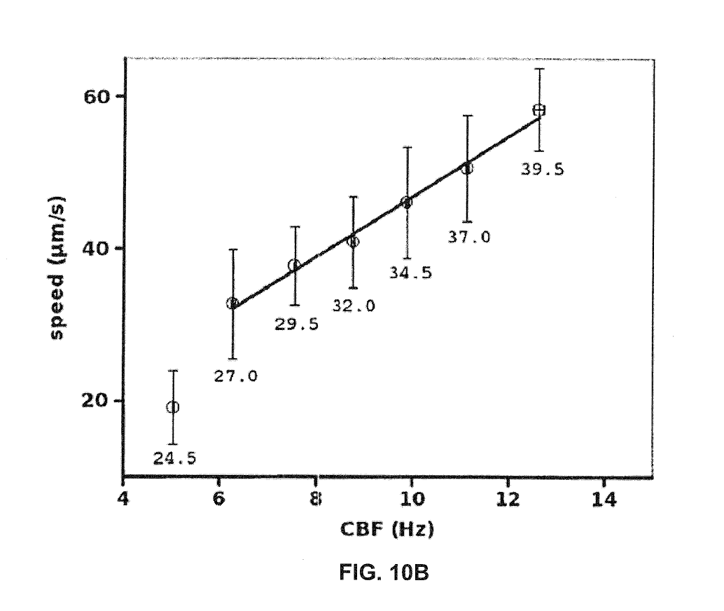

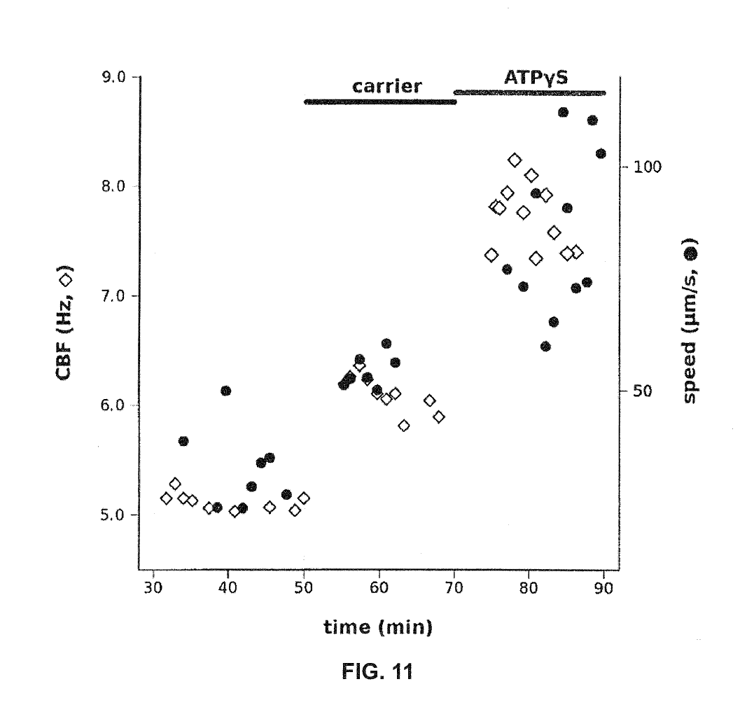

FIG. 10A shows a representative experiment in which the temperature of the HAE cell culture was raised in a step-wise fashion and the CBF and MCT were measured at each step (after the temperature had stabilized), demonstrating that with increasing CBF, the rate of MCT also increases. As reflected in FIG. 10A, a culture in the inventive MCT culturing device demonstrating continuous circular MCT was incubated at a series of increasing temperatures in a step-wise fashion from 24.5° C. to 39.5° C. Each point illustrated in FIG. 10A represents the speed of a single particle (dots) or the average CBF (diamonds) determined from a high-speed video. The data shown is from a single experiment. The linear relationship between CBF and MCT is clearly illustrated in FIG. 10B, in which the data from FIG. 10A is replotted.

Table 2 shows the relationship between CBF and MCT speed as determined from three different experimental protocols (sd=standard deviation; n=number of experiments). Overall, the inventors found an almost linear relationship between ciliary beat frequency and the rate of mucus transport, with an increase of 1 Hz in CBF corresponding to an increase of approximately 8 μm/sec.

TABLE 2
Dependence of mucus speed on CBF ((μm/s)/Hz)

| | 37→25° C. | 0→37° C. | 25→37 in steps |
|---|---|---|---|
| mean | 9.5 | 15.0 | 7.4 |
| sd | 5.5 | 9.1 | 4.2 |
| n | 3 | 8 | 6 |

### Example 4

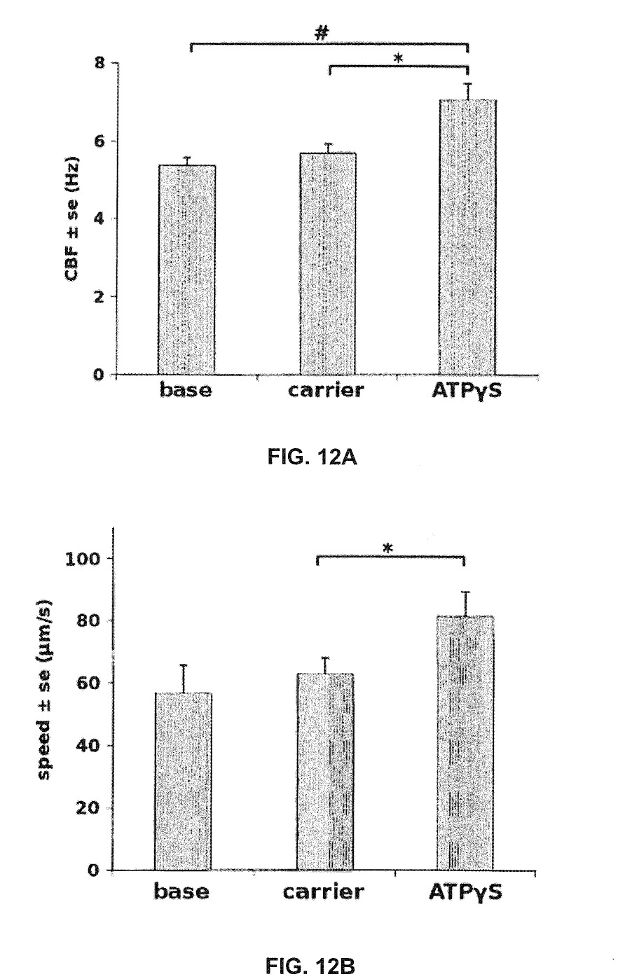

To further characterize the utility of the MCTD as a screening assay, we have begun to investigate the response of CBF and MCT to different test agents. FIG. 11 illustrates changes in CBF and MCT speed in response to stimulation by ATPγS. FIG. 11 illustrates an experiment in which an HAE cell culture displaying circular MCT was first treated with a small amount of buffer followed by the addition (to the apical surface) of an equal amount of buffer containing 1 mM ATPγS. Both CBF and MCT speed increased following treatment with ATPγS. Although both the addition of carrier and the addition of ATPγS increased CBF and MCT, averaging the results from four different experiments/donors (codes) revealed that only the increase caused by ATPγS was significant under these conditions (FIGS. 12A and 12B). FIGS. 12A and 12B illustrate the effect of ATPγS on CBF and MCT in cultures of human airway epithelial cells. Both CBF and MCT were significantly increased in cultures treated with ATPγS (* indicates significantly different from carrier, p<0.05; # indicates significantly different from baseline, p<0.05).

Clearly, the addition of fluid to the apical surface of the MCTD (as in these pilot studies) can affect the measurement of CBF and MCT. According to some embodiments, an aerosol system can be used to deliver test agents to the MCTD (e.g., to its apical surface). These studies provide additional evidence that the MCTD can be useful to measure the effect of different agents on MCT.

### Example 5

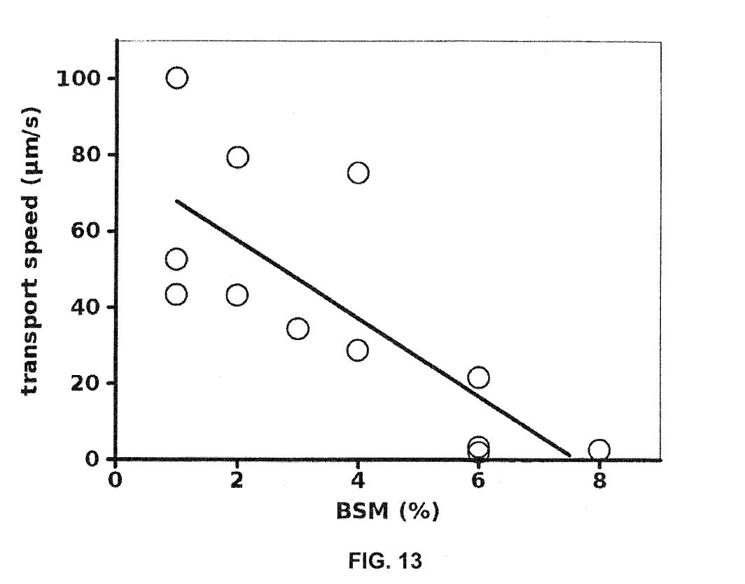

FIG. 13 shows the results of experiments wherein HAE cultures were grown in the MCTD, washed to remove the endogenous mucus, and then incubated with Bovine Salivary Mucin (BSM) at different concentrations. The speed of the MCT in each culture was measured by tracking the movement of particles in or on the BSM around the track of the MCTD.

It will be appreciated that MCT culturing devices and methods according to embodiments of the present invention can provide a number of advantages.

The inventive MCT culturing device (MCTD) and system can take advantage of the tendency of HBE cells to spontaneously organize their beating in the direction of least resistance. By integrating a circular track into an ALI culture system (Gray, Guzman et al. 1996; Fulcher, Gabriel et al. 2005), the inventors have demonstrated that cultures of HBE cells can be regularly generated that exhibit coordinated MCT around the entire circular track. This directed system of MCT has many advantages over the earlier studies of random areas of MCT. For example, in the MCTD, all cultures transport continuously over the same distance and diameter, making measurements of MCT easier to standardize and compare. In addition, because the mucus is transported in a continuous circle, there is no accumulation in the center of the “hurricane”, which can lead to large variations in the thickness (height) of the mucus from the edge to the center. Because the direction and distance of transport in the MCTD is maintained, cultures can be washed free of endogenous mucus and test solutions can be studied. Finally, replicate cultures from the same donor sample can be studied under the same conditions, and the same culture can be studied multiple times.

The inventive MCT culturing device can be used in an in vitro assay suitable for screening novel therapeutics designed to improve MCC in CF patients. The availability of a robust screening system is clearly an aid to the development of new therapeutic agents, as evidenced by the rapid progress towards obtaining small molecule correctors of CFTR function enabled by the development of high-throughput screening programs (Pedemonte, Lukacs et al. 2005).

The MCT culturing device can provide a cell culture device that reproducibly generates MCT in cultures of ciliated epithelial cells. The MCT culturing device can be designed to make the device amenable to studies/measures of MCT.

The MCT culturing device can be constructed to have a standardized geometry to allow for high throughput measures of MCT, for the purpose of screening drugs for their effect on MCT or for any other purpose that would benefit from the ability to make measures of MCT. The MCT culturing device can be scaled down to fit in standard cell culture wells of 6, 12, 24, 48, 96, 384, and possibly other formats.

The MCT culturing device can be used with human ciliated cells as well as with ciliated cells from other species, notably dogs, mice, and pigs.

The MCT culturing device can be sensitive to manipulations that are known to have an effect on MCT, and therefore the device can be used for screening drugs/agents that may increase/improve MCC.

Various modifications may be made to the MCT culturing devices as disclosed herein in accordance with embodiments of the invention. For example, rather than providing the spacer feet 112 that rest on the floor of the vessel 50, the MCT culturing device 100 can be suspended in the cell culture media 52 by hanging the MCT culturing device 100 from the sides of the surrounding vessel 50, or by any other suitable technique that will elevate the membrane 130 above the floor of the vessel 50.

While the device 100 is shown and described as including a discrete inner member 120 mounted within an outer member 110 and supported by a separately formed floor or membrane 130, other constructions may be employed, in accordance with embodiments of the invention, depending on the required functionality of the device. For example, the inner and outer annular walls defining the channel may be formed as a unit with the membrane installed therein. In some embodiments, the inner and outer walls and the floor may all three be formed as a monolithic or unitary structure (i.e., in a case where the floor is not a membrane). Accordingly, the constructions shown in FIGS. 1-4 and disclosed in this paragraph are not exhaustive of the possible constructions.

The materials used to make the MCT culturing device may also be optimized. This may include the material used to make the members 110, 120 themselves and any coatings used to improve the MCT properties of the MCT culturing device. This may also include the porous cell culture membrane 130 and any coatings to improve cell attachment and growth.

The MCT culturing device may include additional features to facilitate the rapid and reproducible measurement of MCT. This may include an integrated numbering/bar coding system, an integrated scale bar in visible and/or fluorescent materials, and/or indentations or other alignment keys to allow the MCT culturing devices to be positioned reproducibly in the vessel 50 or other instruments for the measurement of MCT, the delivery of drugs, the automated changing of media or solutions, etc. Scale markings on the MCT culturing device may be used to automate the measurements.

By way of example, the MCT culturing device 100 as illustrated in FIGS. 1-4 includes reference indicia 150 (FIG. 1; e.g., etched or printed) on the top rim of the outer member 110 forming a scale bar 152. The MCT culturing device 100 as illustrated also includes a series of visible or fluorescent tracking lines 154 on the floor member 130. These indicia may be used to assist in measuring the transport of endogenous mucus or fluorescent particles, for example.

As discussed above, the cell culturing procedure may include artificially creating flow to improve or direct the orientation of the cilia.

The cell culture system 10 and methods may include a prescribed culture medium formulation 52.

A prescribed or standardized “test mucin solution” could be provided to users interested in measuring MCT under a defined set of conditions.

In some embodiments, the MCT culturing device is used to culture human lung epithelial cells. In some embodiments, the MCT culturing device is used to culture human brain ependymal cells or human ciliated oviduct cells. The MCT culturing device may be used to culture dog, mouse, pig, frog, or other vertebrate animal epithelial cells.

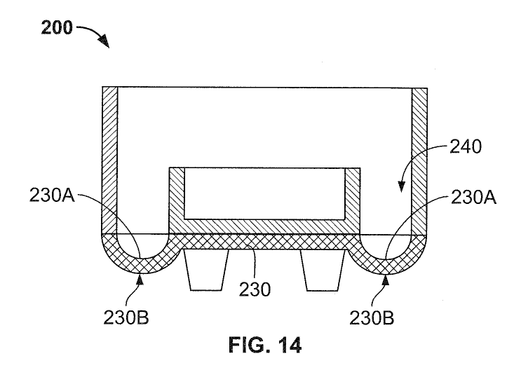

With reference to FIG. 14, an MCT culturing device 200 according to further embodiments is shown therein in cross-section. The device 200 corresponds to and may be used in the same manner as the device 100 except that the portion 230B of the floor member 230 (e.g., a membrane) forming a portion of the channel 240 has an engagement surface 230A that is curved or arcuate in cross-section. According to some embodiments and as illustrated, the cross-sectional profile is concave and, in some embodiments, substantially U-shaped. The surface 230A thus forms an annular or toroidal half-pipe.

In use, the ciliated cells C may be plated on the surface 230A as described above with regard to the surface 130A and, in some embodiments, such that the cells C extend along the bottom of the “U” of the channel and up the side walls of the floor member 230. The curved profile configuration may enhance the development of MCT and/or create a more narrow, central channel, at a specified distance, that may make it easier to perform uniform measurements. In some embodiments, the curved profile floor surface 230A replicates or simulates the shape of a trachea (e.g., a human trachea).

The following references are hereby incorporated herein by reference and are referenced in the description above.
Aitken, M. L., G. Bellon, et al. (2012). “Long-term inhaled dry powder mannitol in cystic fibrosis: an international randomized study.” American Journal of Respiratory and Critical Care Medicine 185(6): 645-652.
Bennett, W. D., M. A. Almond, et al. (2006). “Effect of salmeterol on mucociliary and cough clearance in chronic bronchitis.” Pulm Pharmacol Ther 19(2): 96-100.
Boucher, R. C. (2003). “Regulation of airway surface liquid volume by human airway epithelia.” Pflugers Arch 445(4): 495-498.
Boucher, R. C. (2007). “Airway surface dehydration in cystic fibrosis: pathogenesis and therapy.” Annu Rev Med 58: 157-170.
Boucher, R. C. (2007). “Evidence for airway surface dehydration as the initiating event in CF airway disease.” J Intern Med 261(1): 5-16.
Button, B., L. H. Cai, et al. (2012). “A periciliary brush promotes the lung health by separating the mucus layer from airway epithelia.” Science 337(6097): 937-941.
Davis, C. W. and B. F. Dickey (2008). “Regulated airway goblet cell mucin secretion.” Annu Rev Physiol 70: 487-512.
Donaldson, S. H., W. D. Bennett, et al. (2006). “Mucus clearance and lung function in cystic fibrosis with hypertonic saline.” N Engl J Med 354(3): 241-250.
Donaldson, S. H., T. E. Corcoran, et al. (2007). “Mucociliary clearance as an outcome measure for cystic fibrosis clinical research.” Proc Am Thorac Soc 4(4): 399-405.
Fahy, J. V. and B. F. Dickey (2010). “Airway mucus function and dysfunction.” N Engl J Med 363(23): 2233-2247.
Foster, W. M., D. M. Walters, et al. (2001). “Methodology for the measurement of mucociliary function in the mouse by scintigraphy.” J Appl Physiol 90(3): 1111-1117.
Fulcher, M. L., S. Gabriel, et al. (2005). “Well-differentiated human airway epithelial cell cultures.” Methods Mol Med 107: 183-206.
Gray, T. E., K. Guzman, et al. (1996). “Mucociliary differentiation of serially passaged normal human tracheobronchial epithelial cells.” Am. J. Respir. Cell Mol. Biol. 14: 104-112.
Grubb, B. R., J. H. Jones, et al. (2004). “Mucociliary transport determined by in vivo microdialysis in the airways of normal and CF mice.” Am J Physiol Lung Cell Mol Physiol 286(3): L588-595.
Hirsh, A. J., J. Zhang, et al. (2008). “Pharmacological properties of N-(3,5-diamino-6-chloropyrazine-2-carbonyl)-N′-4-[4-(2,3-dihydroxypropoxyl)phenyl]butyl-guanidine methanesulfonate (552-02), a novel epithelial sodium channel blocker with potential clinical efficacy for cystic fibrosis lung disease.” The Journal of pharmacology and experimental therapeutics 325(1): 77-88.
Hua, X., K. L. Zeman, et al. (2010). “Noninvasive real-time measurement of nasal mucociliary clearance in mice by pinhole gamma scintigraphy.” J Appl Physiol 108(1): 189-196.
Kondo, M., W. E. Finkbeiner, et al. (1991). “A simple technique for culture of highly differentiated cells from dog tracheal epithelium.” Am. J. Physiol. (Lung Cell. Mol. Physiol. 5) 261: L106-L117.
Matsui, H., B. R. Grubb, et al. (1998). “Evidence for periciliary liquid layer depletion, not abnormal ion composition, in the pathogenesis of cystic fibrosis airways disease.” Cell 95(7): 1005-1015.
Matsui, H., S. H. Randell, et al. (1998). “Coordinated clearance of periciliary liquid and mucus from airway surfaces.” J Clin Invest 102(6): 1125-1131.
Morse, D. M., J. L. Smullen, et al. (2001). “Differential effects of UTP, ATP, and adenosine on ciliary activity of human nasal epithelial cells.” Am J Physiol Cell Physiol 280(6): C1485-1497.
Ostrowski, L. E., W. Yin, et al. (2010). “Conditional deletion of dnaic1 in a murine model of primary ciliary dyskinesia causes chronic rhinosinusitis.” Am J Respir Cell Mol Biol 43(1): 55-63.
Pedemonte, N., G. L. Lukacs, et al. (2005). “Small-molecule correctors of defective DeltaF508-CFTR cellular processing identified by high-throughput screening.” J Clin Invest 115(9): 2564-2571.
Rancourt, R. C., S. Tai, et al. (2004). “Thioredoxin liquefies and decreases the viscoelasticity of cystic fibrosis sputum.” American journal of physiology. Lung cellular and molecular physiology 286(5): L931-938.
Ribeiro, C. M., H. Hurd, et al. (2009). “Azithromycin treatment alters gene expression in inflammatory, lipid metabolism, and cell cycle pathways in well-differentiated human airway epithelia.” PLoS One 4(6): e5806.
Sabater, J. R., A. Wanner, et al. (2002). “Montelukast prevents antigen-induced mucociliary dysfunction in sheep.” Am J Respir Crit Care Med 166(11): 1457-1460.
Salathe, M. (2007). “Regulation of mammalian ciliary beating.” Annu Rev Physiol 69: 401-422.
Sisson, J. H., J. A. Stoner, et al. (2003). “All-digital image capture and whole-field analysis of ciliary beat frequency.” J Microsc 211(Pt 2): 103-111.
Suk, J. S., N. J. Boylan, et al. (2011). “N-acetylcysteine Enhances Cystic Fibrosis Sputum Penetration and Airway Gene Transfer by Highly Compacted DNA Nanoparticles.” Mol Ther.
Tarran, R. (2004). “Regulation of airway surface liquid volume and mucus transport by active ion transport.” Proc Am Thorac Soc 1: 42-46.
Tarran, R. and R. C. Boucher (2002). “Thin-film measurements of airway surface liquid volume/composition and mucus transport rates in vitro.” Methods Mol Med 70: 479-492.
Wagener, J. S. and O. Kupfer (2012). “Dornase alfa (Pulmozyme).” Current opinion in pulmonary medicine 18(6): 609-614.
Worthington, E. N. and R. Tarran (2011). “Methods for ASL measurements and mucus transport rates in cell cultures.” Methods Mol Biol 742: 77-92.
You, Y., E. J. Richer, et al. (2002). “Growth and differentiation of mouse tracheal epithelial cells: selection of a proliferative population.” Am J Physiol Lung Cell Mol Physiol 283(6): L1315-1321.
Zhou, Z., D. Treis, et al. (2008). “Preventive but not late amiloride therapy reduces morbidity and mortality of lung disease in betaENaC-overexpressing mice.” Am J Respir Crit Care Med 178(12): 1245-1256.

That which is claimed is:

1. A cell culture assembly comprising:
a cell culturing device including at least one member defining an endless, substantially elliptical channel; and
a layer of ciliated cells disposed in the elliptical channel.

2. The cell culture assembly of claim 1 wherein the cell culturing device includes a semipermeable membrane forming a floor of the channel and supporting the layer of ciliated cells.

3. The cell culture assembly of claim 2 wherein the membrane is porous.

4. The cell culture assembly of claim 3 including culture medium contacting the layer of ciliated cells through the membrane.

5. The cell culture assembly of claim 2 wherein the membrane is formed of a material selected from the group consisting of polycarbonate, mixed cellulose esters, polyester and polytetrafluoroethylene (PTFE).

6. The cell culture assembly of claim 2 including reference indicia on the membrane to assist in measuring a rate of ciliary transport by the layer of ciliated cells.

7. The cell culture assembly of claim 1 including a side wall and reference indicia on the side wall to assist in measuring a rate of ciliary transport by the layer of ciliated cells.

8. The cell culture assembly of claim 1 including at least one tracking bead on the layer of ciliated cells to assist in measuring a rate of ciliary transport by the layer of ciliated cells.

9. The cell culture assembly of claim 1 configured such that the ciliated cells spontaneously orient themselves to transport a material on the layer of ciliated cells in a continuous pattern around the elliptical channel.

10. The cell culture assembly of claim 1 wherein the layer of ciliated cells is a layer of ciliated airway epithelial cells.

11. The cell culture assembly of claim 10 wherein the layer of ciliated airway epithelial cells is a layer of human bronchial epithelial cells.

12. The cell culture assembly of claim 10 including a layer of a transport fluid on top of the layer of ciliated airway epithelial cells.

13. The cell culture assembly of claim 12 wherein the transport fluid is mucus.

14. The cell culture assembly of claim 1 wherein the channel has a substantially uniform width about its full circumference.

15. The cell culture assembly of claim 1 wherein:
the cell culturing device includes a floor of the channel supporting the layer of ciliated cells; and
the floor has a curved cross-sectional profile.

16. A method for culturing ciliated cells, the method comprising:
providing a cell culturing device including at least one member defining an endless, substantially elliptical channel; and
placing a layer of ciliated cells in the elliptical channel.

17. The method of claim 16 wherein the cell culturing device includes a semipermeable membrane forming a floor of the channel, and the method includes placing the layer of ciliated cells on the membrane such that the layer of ciliated cells is supported thereby.

18. The method of claim 17 including providing a culture medium in the cell culturing device and contacting the layer of ciliated cells with the culture medium through the membrane.

19. The method of claim 16 wherein the ciliated cells spontaneously orient themselves to transport a material on the layer of ciliated cells in a continuous pattern around the elliptical channel.

20. The method of claim 16 including using the cell culturing device to screen, test, examine, and/or compare the effectiveness of one or more drugs and/or chemical agents for their effect on improving mucociliary transport by the layer of ciliated cells.

21. A cell culturing device for culturing a layer of cells, the cell culturing device comprising:
at least one member defining an endless, substantially elliptical channel; and
a semipermeable membrane forming a floor of the channel to receive and support the layer of cells.

22. The cell culturing device of claim 21 configured such that, when the layer of cells is a layer of ciliated cells, the cell culturing device causes the ciliated cells to spontaneously orient themselves to transport a material on the layer of cells in a continuous pattern around the elliptical channel.

23. A method for culturing ciliated cells comprising:
culturing ciliated cells in a cell culturing device; and
using the cell culturing device to screen, test, examine, and/or compare the effectiveness of one or more drugs and/or chemical agents for their effect on improving mucociliary transport by the layer of ciliated cells.
